(19) United States
(12) Patent Application Publication
(10) Pub. No.: US 2018/0349828 A1
(43) Pub. Date: Dec. 6, 2018

(54) PEOPLE INTERRUPTION MANAGEMENT SYSTEM AND METHOD BASED ON TASK DETECTION AND PHYSIOLOGICAL MEASURES

(71) Applicant: International Business Machines Corporation, Armonk, NY (US)

(72) Inventors: Marco Aurelio STELMAR NETTO, SAO PAULO (BR); Vagner Figueredo DE SANTANA, SAO PAULO (BR)

(21) Appl. No.: 15/615,052

(22) Filed: Jun. 6, 2017

(51) Int. Cl. G06Q 10/06; A61B 5/01; A61B 5/02; A61B 3/11

(52) U.S. Cl. CPC G06Q 10/06398; G06Q 10/06395; A61B 3/11; A61B 5/02; A61B 5/01

(57) ABSTRACT

A computer-implemented method, an interruption management system and computer program product automatically determine when a user should not be interrupted in a collaborative work. An event is determined to be occurring. Physiological data of the user is captured. Complex task steps of the event are identified. When the user is determined to be performing a complex task step and the physiological data of the user is determined to be an outlier, a notification indicating that the user should not be interrupted is output to an interruptor indicator. The captured physiological data may include eye dilation, skin conductance, heart rate, eye tracking, glomerular filtration rate and skin temperature. The event may be pair programming, software support, hardware support, medical surgery, human-robot interaction or command center operation. The physiological data is determined to be an outlier using standard deviation. The notification is output as a visual indicator.

### BACKGROUND

The present disclosure generally relates to interruption management in collaborative activities and more particularly relates to a system and method for managing people interruption based on task detection and physiological measures.

Several problems are solved when multiple people collaborate. Examples of collaborative activities may include handling machines for manufacturing, analyzing data for insights and decision making, software development, among others. Depending on the level of attention and the currently activity a person is pursuing, the interruption of another person who is involved in the collaborative work can have a negative impact. People naturally take momentary breaks, so there are periods of time where interruptions are less intrusive or much more damaging or distracting. However, there are no reliable methods to determine when an undesirable interruption should be avoided, particularly from a remote location.

### BRIEF SUMMARY

In various embodiments, a computer-implemented method, an interruption management system and a computer program product for automatically determining when a user should not be interrupted in a collaborative work are disclosed. The method comprises determining that an event is occurring, capturing physiological data of the user, identifying complex task steps of the event, determining that the user is performing a complex task step, determining that the physiological data of the user is an outlier and outputting a notification indicating that the user should not be interrupted.

### BRIEF DESCRIPTION OF THE SEVERAL VIEWS OF THE DRAWINGS

The accompanying figures where like reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a block diagram illustrating one example of an operating environment comprising an interruption management system according to one embodiment of the present invention;

FIG. 2 is a block diagram of one example of logical components of an interruptor manager according to one embodiment of the present invention;

FIG. 3 is an operational flow diagram illustrating one process of managing interruption based on task detection and physiological measures according to one embodiment of the present invention; and

FIG. 4 is a block diagram of hardware components of an interruptor manager according to one embodiment of the present invention.

### DETAILED DESCRIPTION

Embodiments of the present invention provide a system and associated methods for a collaborative work setting that identifies the activity a person is executing and his/her concentration level using physiological measures and is able to notify other collaborator peers to do not disrupt that person. This interruption management system can be used in a number of scenarios including manufacturing, pair programming and collaborative data analytics.

Tasks are detected based on logs of events triggered while people carry out their daily workload. Log analysis may be performed to identify critical points in these daily tasks. In addition to logs of events, physiological data is also considered to identify, within the tasks, outlying conditions representing stressful situations during which the person should not be interrupted. Physiological data may include, for example, eye dilation, skin conductance, heart rate, eye tracking, glomerular filtration rate, skin temperature, etc. When a critical and stressful situation is detected, the system informs other people to avoid interrupting the person carrying out the work.

Examples of collaborative work that require attention where interruptions at unsuitable times may have negative impact include pair programming, software and hardware support, medical surgeries, human-robot interaction and command center operation. Pair programming is software development performed by multiple people. Interruptions may be especially disruptive at moments when an important bug fix needs to be created. Software and hardware support involves multiple people working together to solve a hardware and/or software based problem. Interruptions during medical surgeries at a delicate moment could be fatal for the patient. During human-robot interaction, there are a number of contexts, based on common sense, where humans should not be interrupted. For human-robot interaction, there is still a challenge in implementing this “common sense” for the robot. Embodiments of this invention allow the robot to understand when it would not be an appropriate time to interrupt a human for collaborative interaction by understanding the task the human is performing and observing the human's physiological measurements. In a command center (e.g., systems service, city hall command center, etc.), when a crisis situation occurs, multiple specialists are working against the clock in order to solve complex tasks, so interruptions that increase response time may cause considerable impact.

Operating Environment

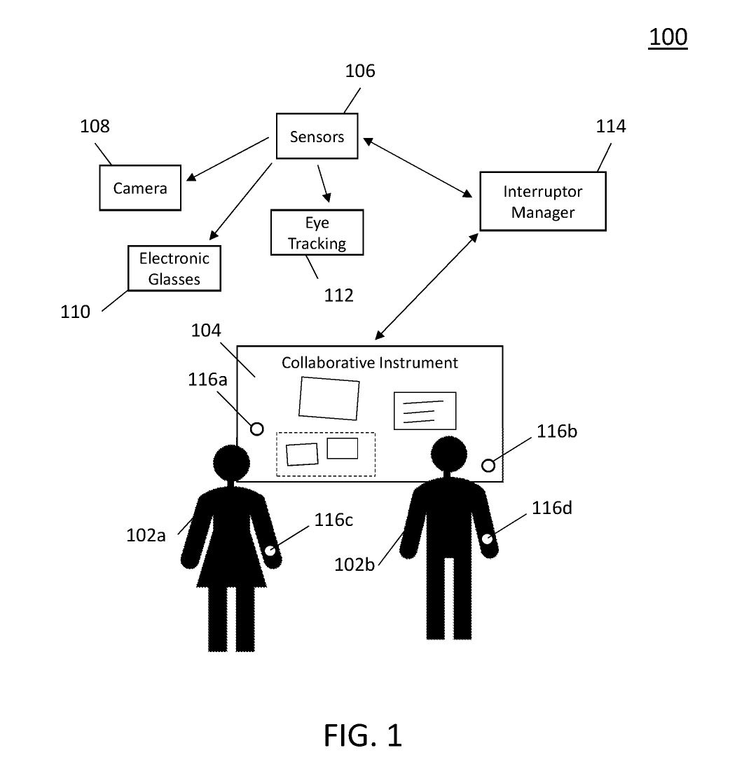

Turning now to FIG. 1, a block diagram of one example of an operating environment comprising an interruption management system 100 according to one embodiment of the present invention. A multiplicity of human users 102a, 102b (referenced generally or collectively herein as “user 102”) interact with the interruption management system 100 to perform a collaborative work. The users 102 interact with the system 100 through a collaborative instrument 104, such as a manufacturing machine(s), a computer(s), a whiteboard(s), sensors monitoring a patient, etc. While the users 102 are using the collaborative instrument 104, various sensors 106 capture physiological data and contextual activity associated with the user 102. For example, sensors may include electronic devices such as a camera 108, electronic glasses 110, other eye tracking systems 112, a heartrate monitor (not shown), brain activity sensors (not shown), etc., that capture information from which a level of concentration for the user 102 may be ascertained.

The sensors 106 send the captured information to an interruptor manager 114 uses the information to determine whether the user 102 may or may not be interrupted at any given moment. The interruptor manager 114 may be implemented using a dedicated computer (i.e. an interruption management server) programmed to interact with the sensors 106, the collaborative instrument 104 and various interruptor indicators 116 (four shown 116a, 116b, 116c, 116d). The interruptor indicators are referenced generally or collectively herein as “interruptor indicator 116.” The interruptor indicators 116 may be a visual indicator, such as a light-emitting diode (LED), a light, a computer graphic, a wrist band, a watch or other device (such as a mobile device running a mobile application), which may be activated to notify other people that the particular user may not be interrupted at the present time.

In addition, the interruptor manager 114 may be in communication with remote users of the interruption management system 100 via a wired or wireless network (not shown). The interruptor manager 114 may activate remote indicators, such as an icon or other computer graphic, on the remote user's computer to indicate that a certain user 102 may not be currently interrupted.

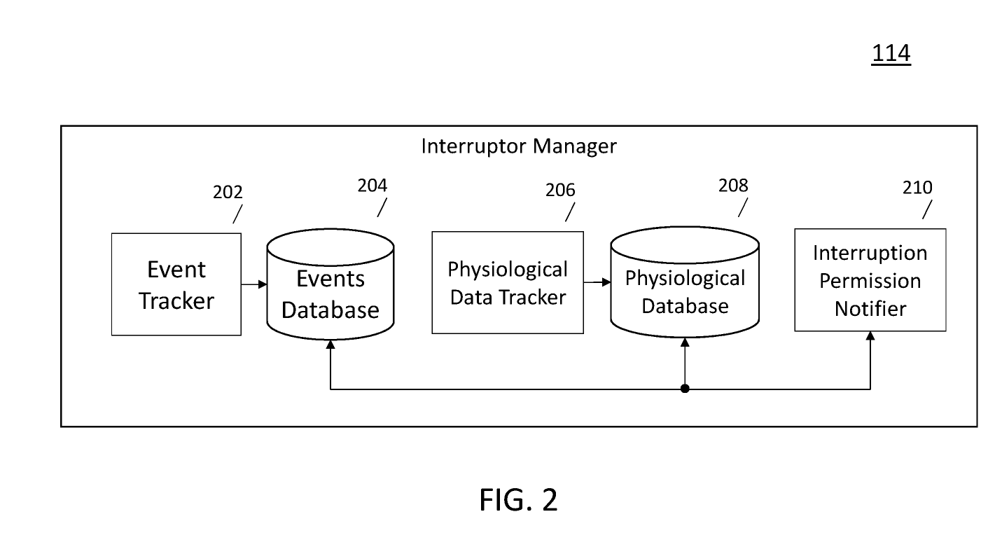

FIG. 2 depicts a block diagram of an example interruptor manager 114 in accordance with the present invention. Interruptor manager 114 includes an event tracker 202 coupled with an events database 204, a physiological data tracker 206 coupled with a physiological database 208, and an interruption permission notifier 210. In some embodiments, the events database 204 and/or the physiological database 208 may be contained within the interruptor manager 114, while in other embodiments, the events database 204 and/or the physiological database 208 may be collocated with the interruptor manager 114 or remotely located from the interruptor manager 114 and accessible via a network connection (not shown).

The event tracker 202 monitors events requiring a predetermined concentration level by a user 102 of the collaborative instrument 104. For example, when the collaborative instrument 104 is a machine, the event tracker 202 may monitor physical activities such as turning a knob or handle, pushing buttons, adjusting the temperature, whether access doors are open, replacement of parts, etc. When the collaborative instrument 104 is a computer, the event tracker 202 may monitor events such as visualizing data containing X number of points, fixing computer bugs, renting cloud resources, etc. When the collaborative instrument 104 is used in a medical setting, the event tracker 202 may monitor operations such as cutting or connecting tissues. The event tracker 202 logs all events in the events database 204.

The physiological data tracker 206 monitors information captured by the sensors 106 concerning physiological conditions of the user 102. The physiological conditions may include the user's heart rate, eye movement, pupil dilation, skin conductance, etc. The physiological data tracker 206 logs the physiological data of the user 102 in the physiological database 208. The interruption permission notifier 210 analyzes the events stored in the events database 204 and the physiological data stored in the physiological database 208 using the methods described below and notifies others that the user 102 should not be interrupted at the present moment by activating one or more of the interruptor indicators 116.

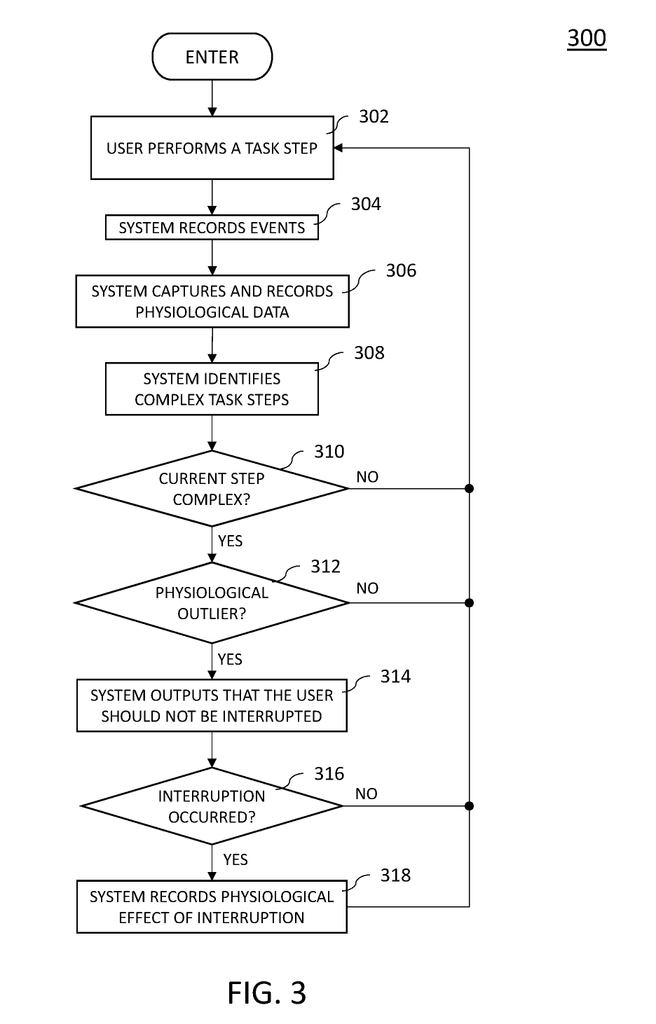

Exemplary steps for operating an interruption management system 100, in accordance with the present invention, will be described with reference to FIG. 3. FIG. 3 depicts an operational flowchart 300 for performing an exemplary process according to one embodiment of the present invention. At step 302, the interruption management system 100 recognizes that a certain user 102 is performing a task step using the collaborative instrument 104. Any task performed by the user 102 in a system that triggers interaction events can be considered in accordance with the present invention. For example, a task step may include low level interaction data triggered during graphical user interface (GUI) interactions such as mouse movements or clicks, pressed keys, a touch sensed on a touch screen display, etc., to multi-modal interaction commands including voice commands, gesture interaction, haptic events, etc. At step 304, the system 100 logs the triggered event in the events database 204.

At step 306, the system 100 captures physiological data and records this data in the physiological database 208. Physiological data may include pupil dilation, pupil mean diameter, eye fixation time, eye blink rate, eye scanning range, skin conductance, heart rate, and any other physiological data that is highly correlated with cognitive workload which allows the system 100 to identify when and when not to interrupt the user performing the task at hand.

At step 308, the system 100 identifies complex task steps. Step 308 can be performed using multiple approaches. For instance, when building a usage graph structure where each node represents an action or step in a directed graph, it is possible to identify complex task steps from cyclic actions. In a usage graph, each event-target pair is represented as a node, that in turn is part of a directed graph. Hence, repeated event-target pairs during a task depict cyclic actions. By identifying outliers in terms of time taken or number of actions taken to reach a certain node in the usage graph structure, it is possible to identify complex task steps.

Another possibility is to use graph mining metrics to identify complex steps, for instance, nodes with a high betweenness metric value or nodes with high eccentricity values. A third possibility for detecting complex tasks is to use a state machine to identify transitions that involve more steps or take more time or cyclic actions that indicate errors or multiple attempts to conclude a task.

At step 310, a comparison is performed between the current task step and steps identified as complex task steps in order to verify whether the current step is marked as a complex step. If the current task step is not deemed a complex step, the process returns to step 302 to continue monitoring user-performed tasks. However, if the current task step is deemed a complex step, at step 310, the interruption permission notifier 210 of the interruptor manager 114 determines whether the captured physiological data is an outlier, at step 312. Outliers can be computed in multiple ways. For example, by using methods based on standard deviation, classification, clustering, nearest neighbor, statistics, information theory, spectral, or other known methods. If the physiological data is not an outlier, the process returns to step 302 to continue monitoring user-performed tasks. However, if the physiological data is an outlier, at step 312, the system 100 outputs that the user should not be interrupted, at step 314. The output may be in multiple forms using the interruptor indicators 116, for instance, by activating a light or LED, changing an instant messenger status to “Do not disturb,” informing of the status via a mobile application, displaying a notification on a computer screen, etc.

If an interruption does occur, at step 316, during the time when the system 100 warns against interruptions, the system 100 records the physiological effects of the interruption, at step 318, in order to supply the physiological database 208 with references. If an interruption does not occur, at step 316, the process returns to step 302 to continue monitoring user-performed tasks.

Interruption Management Server

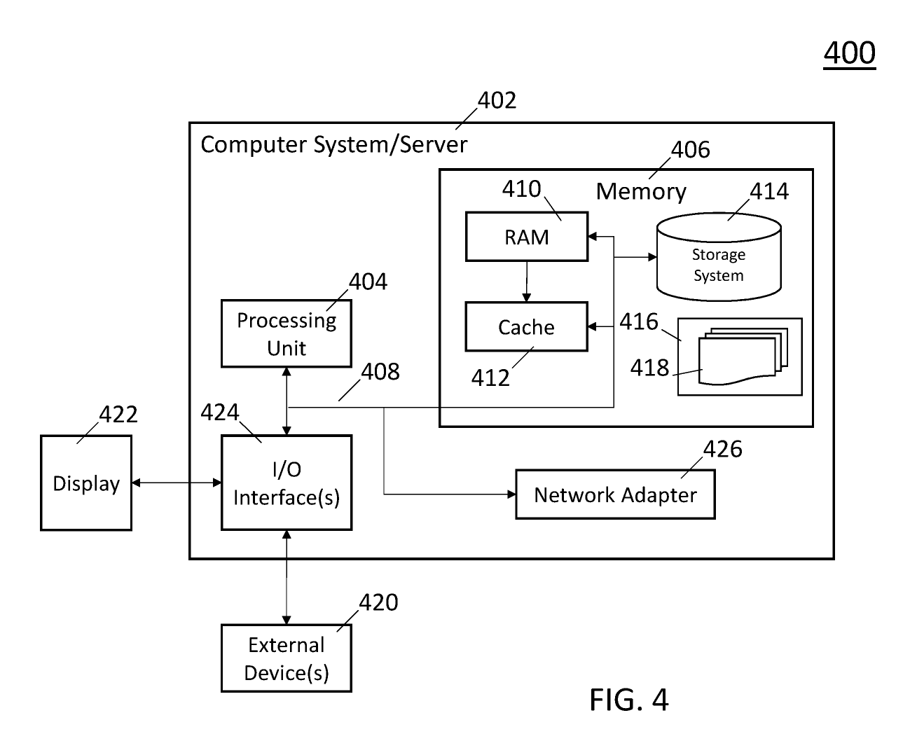

Referring now to FIG. 4, a block diagram illustrating an information processing system 400 that can be utilized in embodiments of the present invention is shown. The information processing system 402 is based upon a suitably configured processing system configured to implement one or more embodiments of the present disclosure (e.g., interruptor manager 114). Any suitably configured processing system can be used as the information processing system 402 in embodiments of the present invention. The components of the information processing system 402 can include, but are not limited to, one or more processors or processing units 404, a system memory 406, and a bus 408 that couples various system components including the system memory 406 to the processor 404.

The bus 408 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Although not shown in FIG. 4, the main memory 406 may include interruptor manager 114, event tracker 202, events database 204, physiological data tracker 206, physiological database 208 and interruption permission notifier 210 shown in FIG. 2. One or more of these components can reside within the processor 404, or be a separate hardware component. The system memory 406 can also include computer system readable media in the form of volatile memory, such as random access memory (RAM) 410 and/or cache memory 412. The information processing system 402 can further include other removable/non-removable, volatile/non-volatile computer system storage media. By way of example only, a storage system 414 can be provided for reading from and writing to a non-removable or removable, non-volatile media such as one or more solid state disks and/or magnetic media (typically called a “hard drive”). A magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 408 by one or more data media interfaces. The memory 406 can include at least one program product having a set of program modules that are configured to carry out the functions of an embodiment of the present disclosure.

Program/utility 416, having a set of program modules 418, may be stored in memory 406 by way of example, and not limitation, as well as an operating system, one or more application programs, other program modules, and program data. Each of the operating system, one or more application programs, other program modules, and program data or some combination thereof, may include an implementation of a networking environment. Program modules 418 generally carry out the functions and/or methodologies of embodiments of the present disclosure.

The information processing system 402 can also communicate with one or more external devices 420 (such as a keyboard, a pointing device, a display 422, etc.); one or more devices that enable a user to interact with the information processing system 402; and/or any devices (e.g., network card, modem, etc.) that enable computer system/server 402 to communicate with one or more other computing devices. Such communication can occur via I/O interfaces 424. Still yet, the information processing system 402 can communicate with one or more networks such as a local area network (LAN), a general wide area network (WAN), and/or a public network (e.g., the Internet) via network adapter 426. As depicted, the network adapter 426 communicates with the other components of information processing system 402 via the bus 408. Other hardware and/or software components can also be used in conjunction with the information processing system 702. Examples include, but are not limited to: microcode, device drivers, redundant processing units, external disk drive arrays, RAID systems, tape drives, and data archival storage systems.

### Non-Limiting Embodiments

As will be appreciated by one skilled in the art, aspects of the present disclosure may be embodied as a system, method, or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit”,” “module”, or “system.”

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method for automatically determining when a user should not be interrupted in a collaborative work, the method comprising:
determining that an event is occurring;
capturing physiological data of the user;
identifying complex task steps of the event;
determining that the user is performing a complex task step;
determining that the physiological data of the user is an outlier; and
outputting a notification indicating that the user should not be interrupted.

2. The method of claim 1, wherein the captured physiological data is at least one of eye dilation, pupil mean diameter, eye fixation time, eye blink rate, eye scanning range, skin conductance, heart rate, eye tracking, glomerular filtration rate, and skin temperature.

3. The method of claim 1, wherein the event is one of pair programming, software support, hardware support, medical surgery, human-robot interaction and command center operation.

4. The method of claim 1, wherein the physiological data is determined to be an outlier using at least one of standard deviation, classification, clustering, nearest neighbor, statistics, information theory, and spectral methods.

5. The method of claim 1, wherein the notification is output as a visual indicator.

6. The method of claim 1, wherein the notification is outputted to at least one of a light-emitting diode (LED), a light, a computer, a wrist band, a watch and a mobile device.

7. The method of claim 1, wherein physiological data is captured using at least one of a camera, electronic glasses, an eye tracking system, brain activity sensors and a heartrate monitor.

8. The method of claim 1, wherein the event is determined to be occurring when the user is interacting with a collaborative instrument.

9. The method of claim 8, wherein the collaborative instrument is one of a manufacturing machine, a computer, a whiteboard and sensors monitoring a patient.

10. An interruption management system comprising:
at least one sensor that captures physiological data of a user;
a collaborative instrument that receives input from a plurality of users;
at least one interruptor indicator; and
an interruptor manager in communication with the at least one sensor, the collaborative instrument and the at least one interruptor indicator, the interruptor manager comprising:
a memory storing computer instructions;
a communication interface; and
a processor, operably coupled with the memory and the communication interface, which:
determines that an event is occurring;
identifies complex task steps of the event;
determines that the user is performing a complex task step;
determines that the physiological data of the user is an outlier; and
outputs a notification to the at least one interruptor indicator that indicates when the user should not be interrupted.

11. The interruption management system of claim 10, wherein the captured physiological data is at least one of eye dilation, pupil mean diameter, eye fixation time, eye blink rate, eye scanning range, skin conductance, heart rate, eye tracking, glomerular filtration rate, and skin temperature.

12. The interruption management system of claim 10, wherein the event is one of pair programming, software support, hardware support, medical surgery, human-robot interaction and command center operation.

13. The interruption management system of claim 10, wherein the processor determines the physiological data is an outlier using at least one of standard deviation, classification, clustering, nearest neighbor, statistics, information theory, and spectral methods.

14. The interruption management system of claim 10, wherein the notification is output as a visual indicator.

15. The interruption management system of claim 10, wherein the notification is outputted to at least one of a light-emitting diode (LED), a light, a computer, a wrist band, a watch and a mobile device.

16. The interruption management system of claim 10, wherein the at least one sensor is at least one of a camera, electronic glasses, an eye tracking system, brain activity sensors and a heartrate monitor.

17. The interruption management system of claim 10, wherein processor determines that the event is occurring when the user is interacting with the collaborative instrument.

18. The interruption management system of claim 10, wherein the collaborative instrument is one of a manufacturing machine, a computer, a whiteboard and sensors monitoring a patient.

19. A computer program product for automatically determining when a user should not be interrupted in a collaborative work, the computer program product comprising:
a non-transitory storage medium, readable by a processing circuit and storing instructions for execution by the processing circuit for performing a method comprising:
determining that an event is occurring;
capturing physiological data of the user;
identifying complex task steps of the event;
determining that the user is performing a complex task step;
determining that the physiological data of the user is an outlier; and
outputting a notification indicating that the user should not be interrupted.

20. The computer program product of claim 19, wherein the event is one of pair programming, software support, hardware support, medical surgery, human-robot interaction and command center operation.
